(19) United States
(12) Patent Application Publication
(10) Pub. No.: US 2009/0264459 A1
(43) Pub. Date: Oct. 22, 2009

(54) Organic Compounds

(76) Inventors: Stephen Paul Collingwood, West Sussex (GB); Robin Alec Fairhurst, West Sussex (GB); Neil John Press, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/308,231

(22) PCT Filed: Jun. 28, 2007
(86) PCT No.: PCT/EP2007/005745

§ 371 Date: Dec. 9, 2008

(30) Foreign Application Priority Data
Jun. 30, 2006 (GB) 0613154.4

(51) Int. Cl. A61K 31/4375; C07D 471/04; C07D 417/14; C07D 417/12; A61K 31/454; A61K 31/428; A61P 11/00

(52) U.S. Cl. 514/300; 546/122; 546/198; 548/157; 514/321; 514/367

(57) ABSTRACT

Compounds of formula (I) in free or a pharmaceutically acceptable salt form, wherein X¹, L and Q have the meanings as indicated in the specification, are useful for treating obstructive or inflammatory airways diseases. Pharmaceutical compositions that contain the compounds and processes for preparing the compounds are also described.

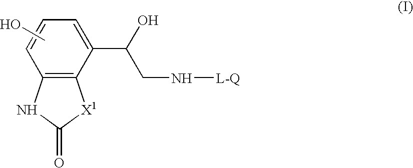

The present invention relates to organic compounds useful as pharmaceutical agents, to pharmaceutical compositions containing such compounds, to their use in manufacturing medicaments, to processes for their preparation and to intermediates useful in their preparation. More specifically, this invention relates to organic compounds possessing activity as β₂ adrenoceptor agonists and optionally as phosphodiesterase inhibitors and/or M₃ muscarinic antagonists.

β₂ adrenergic receptor antagonists have been disclosed as suitable for treating asthma, obstructive pulmonary diseases (such as bronchitis), premature labour, diabetes and the like. Phosphodiesterase inhibitors, particularly PDE-4 inhibitor, are also known for their beneficial effect in the treatment of asthma, obstructive pulmonary diseases (such as bronchitis), rheumatoid arthritis, multiple sclerosis and Crohn's disease. Muscarinic M₃ antagonists have been said to be beneficial in the treatment of pulmonary disease such as bronchonstrictionary asthma and other obstructive or inflammatory airways diseases.

It is desirable to provide compounds that possess β₂-adrenegic receptor antagonists activity optionally together with PDE-4 inhibitory activity and/or M₃ antagonist activity. Such compounds will have numerous pharmaceutical uses including but not limited to treatment of various pulmonary diseases. The active sites of β₂-adrenergice receptors and PDE-4 and M₃ receptors are structurally different so that it is difficult to obtain molecules which inhibit β₂-adrenergic receptors and/or PDE-4 and M₃ receptors. Similarly the structural parameters of β₂-adrenergic receptor antagonists, DPE-4 inhibitors and M3 receptor antagonists which have received detailed investigation have not generally possessed large substituents without adversely effecting activity.

Surprisingly it has been found that it is possible to provide compounds that possess β₂-adrenergic receptor activity optionally together with PDE-4 inhibitory and/or M₃ antagonist activity.

Accordingly, the present invention provides compounds of the formula (I):—

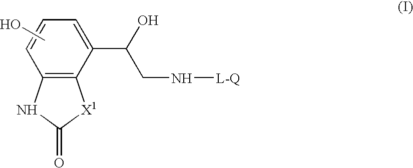

where Q is

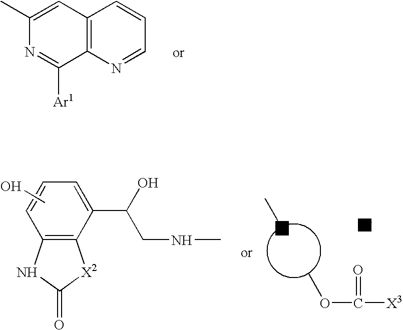

where X³ is

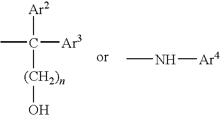

wherein
X¹ and X² are independently selected from S, CH₂CH₂, CH:CH₂ or CH₂0;
Ar¹ is a phenyl, pyridyl, diazinyl or triazinyl group optionally substituted by one or two moieties selected from methyl, ethyl, fluorine, chlorine, bromine, cyano, methoxy, ethoxy, methoxymethyl, trifluoromethyl, trifluoromethoxy, methylthio, ethylthio or methylsulforyl;
Ar² is C₃-C₁₀-cycloalkyl, a thienyl group or a phenyl group optionally substituted by one or two moieties selected from methyl, ethyl, fluorine, chlorine, bromine, cyano, methoxy, ethoxy, methoxymethyl, trifluoromethyl, trifluoromethoxy, methylthio, ethylthio or methylsulforyl; in compounds wherein R¹ is an optionally substituted phenyl group, Ar² may be directly linked to R¹ by a CH₂, CH₂CH₂, CH:CH, OCH₂ or CH₂O group;
Ar³ is C₃-C₁₀-cycloalkyl, a thienyl group or a phenyl group substituted by one or two moieties selected from methyl, ethyl, fluorine, chlorine, bromine, cyano, methoxy, ethoxy, methoxymethyl, trifluoromethyl, trifluoromethoxy, methylthio, ethylthio, methylsulfonyl; in compounds wherein Ar² is an optionally substituted phenyl group R may be linked to Ar² by a CH₂, CH₂CH₂, CH:CH, OCH₂ of CH₂O group.
Ar⁴ is a biphenyl group optionally substituted by one or two moieties selected from fluorine, chlorine, bromine, methyl, ethyl, cyano, methoxy, ethoxy, methoxymethyl, trifluoromethyl, trifluoromethoxy, methylthio, ethylthio or methylsulforyl; n is 0, 1 or 2;
L is a hydrocarbon linking group of 2 to 20 carbon atoms which may be optionally interrupted by O, N, or CO, and is aptly a group of the form:

Y¹—Y²—Y³

wherein:
Y² is not present or is a group of the formula CH—Y⁴-Q or —N—Y⁴-Q where Q independently is a group as defined above and Y⁴ is a bond or a hydrocarbon linking group of 1 to 8 carbon atoms optionally interrupted by O, NH or CO;
Y¹ is attached to the NH group shown is formula (I) and is a hydrocarbon linking group of 2 to 20 carbon atoms optionally containing an ether oxygen atom or;
Y³ is not present or is selected from 0 or a group NR² where R² is a hydrogen atom or an alkyl group of 2 to 3 carbon atoms optionally linked to a carbon atom within Y¹ or Y² to form a ring or 4, 5, 6 or 7 ring atoms;
and

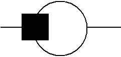

represents a group of the formula:

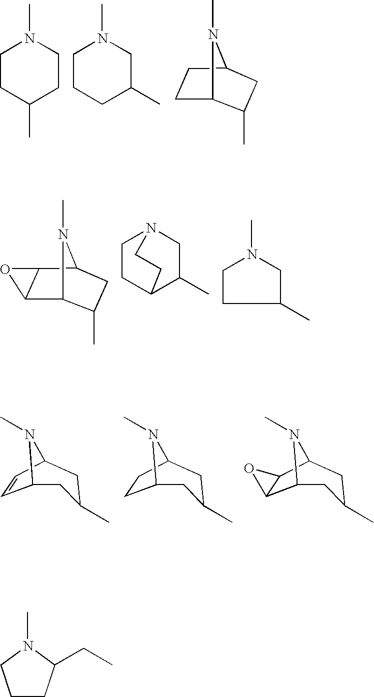

or a pharmaceutically acceptable salt thereof.

The tertiary amine group shown is also optionally alkylated with group NN to give the corresponding quaternary amine. Group NN can be C₁-C₈-alkyl, C₁-C₈-alkaryl, C₁-C₈-alk heteroaryl, CH₂CO-aryl or CH₂CO-heteroaryl.

In certain compounds of the formula (I) L is an alkyl, an alkenyl or alkyl group. Such groups may be straight chained, branched or cyclic or contain combinations of such structures. Such moieties may be interrupted by one or more oxygen atoms, carbonyl groups or amino groups (which may be substituted by alkyl, aryl or aralkyl groups) or by an aryl group, particularly phenyl groups. 1,4-disubstituted.

Rings forming L or part thereof are aptly of 3, 4, 5, 6, 7 or 8 ring members and are favourably of 4, 5 of 6 ring members. The ring members will generally be carbon atoms but rings may contain an oxygen atom or optionally substituted NH groups where the optional substituent is aptly an alkyl group of 1, 2, 3 or 4 carbon atoms.

In certain compounds of the formula (I) apt groups L include:

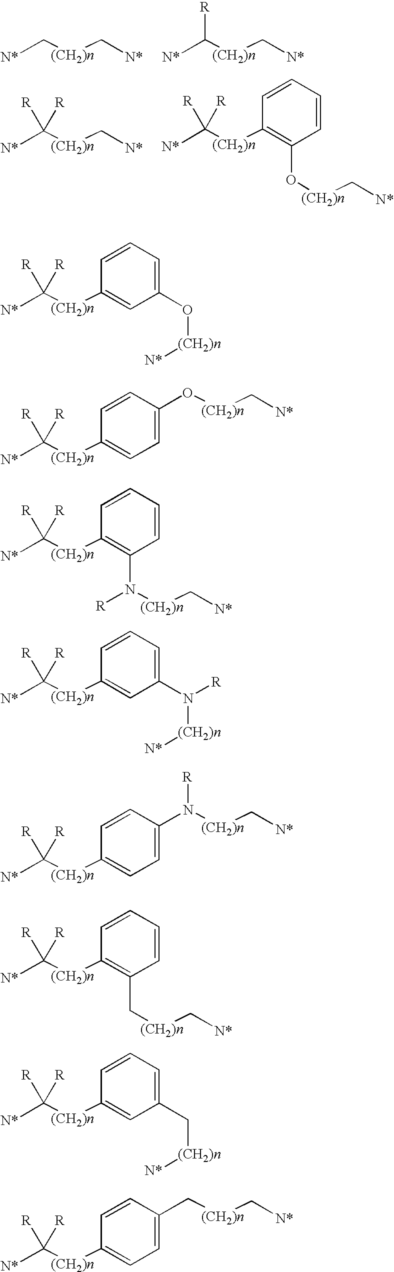

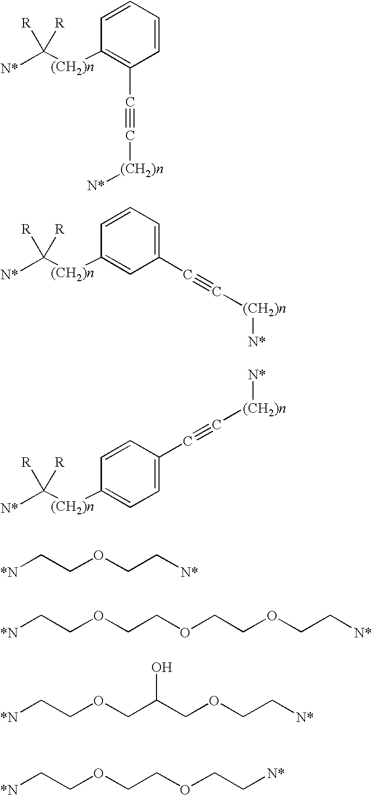

wherein n is 0 to 18, R is hydrogen or alkyl, especially C₁-C₄-alkyl, and N* denotes an available attachment sites.

In certain other compounds of the formula (I) apt groups L include:

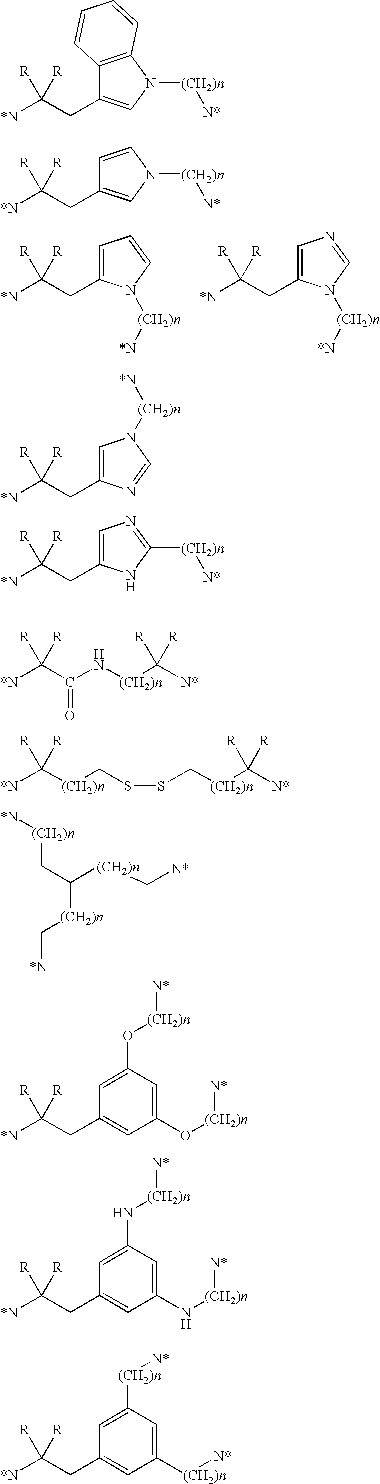

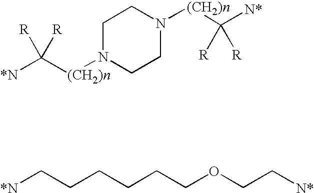

wherein n is 0 to 18, R is hydrogen or alkyl, especially C₁-C₄-alkyl, and N* denotes an available attachment sites.

Preferred compounds of the formula (I) include those having the following general formula:

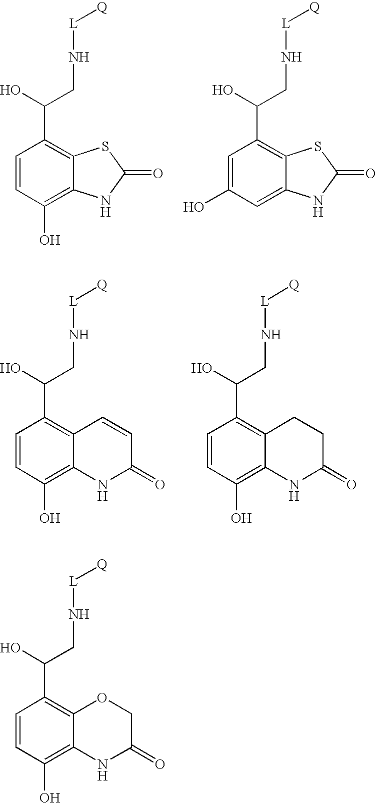

wherein L and Q are as hereinbefore defined.

In certain apt compounds of the formula (I) X¹ is S.

In certain apt compounds of the formula (I) the hydroxyl group of the fused benzene ring is para to the attachment of the side chain.

One sub-set of β₂-adrenergic receptor antagonist compounds of the formula (I) include those of the formula (II)

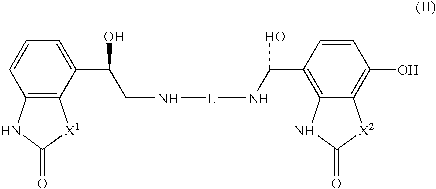

and pharmaceutically acceptable salts thereof wherein X¹, X² and L are as defined in relation to formula (I).

Favourably in the compounds of formula (II) X¹ is sulphur and X² is sulphur.

Particularly apt linker groups L for use, in the compounds of formula (II) include those of the formulas —Y¹—Y³— wherein Y¹ is: a (CH₂)ₘ⁵, (CH₂)ₘ⁶-cyclopentyl or (CH₂)ₘ⁶-cyclohexyl group and Y² is a (CH₂)ₘ⁷ group wherein ₘ⁵ is 1, 2, 3 or 4 and ₘ⁶ is 0, 1, 2 and m⁷ is 1, 2, 3 or 4.

Preferred groups L for use in the compounds of formula (II) include group which is groups of the formula —(CH₂)ₚ— where p is 2, 3, 4, 5, 6, 7 or 8. The cyclohexyl group is also a preferred group L especially the 1,4-para distributed cyclohexyl group.

Particular apt β₂-adrenergic receptor antagonist formula (II) include:
(R)-1-benzothiazol-7-yl-2-[5-(R)-2-benzthiaxol-2-one-7-yl-2-hydroxy-ethylamino]-pentylamino]-ethanol; and
(R)-1-benzothiazol-7-yl-2-[5-(R)-2-benzthiaxol-2-one-7-yl-2-hydroxy-ethylamino]-cyclolexye-ethanol
   and pharmaceutically acceptable salts thereof.

One sub-set of compounds of the formula (I) possessing β₂-adrenergic receptor antagonist and PDE-4 inhibitory properties include those of the formula (III):

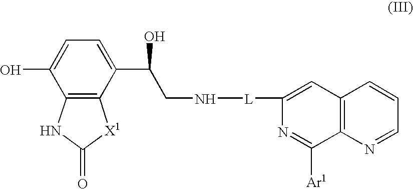

wherein X¹, L and Ar¹ are as defined in relation to formula (I), and pharmaceutically acceptable salts thereof.

In compounds of formula (III) X^{t} is aptly S.

Apt groups L for inclusion in compounds of formula (III) include those of the formula Y¹—Y³ wherein Y₁ is

—(CH₂)ₘ¹—(C₆H₄)ₘ²—

where m¹ is 1, 2, 3, 4, 5 or 6 and m² is 0 or 1;

### and Y² is

—(CH₂)ₘ³—; —CH≡CH(CH₂)ₘ³—; C≡C—(CH₂)ₘ³—O—,

—(CH₂)ₘ³0—, —CH≡CH(CH₂)ₘ³—NH—; —C≡C(CH₂)ₘ³NH—;

—(CH₂)ₘ³NH—; C≡C(CH₂)ₘ³NH—,

-0-(CH₂)ₘ³—, -0(CH₂)ₘ³0-,

-0-(CH₂)ₘ³NH—

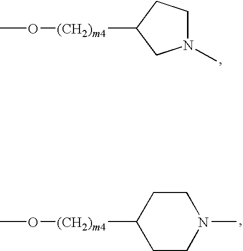

where m³ is 1, 2, 3 or 4; and m⁴ is 0, 1, 2, 3 or 4.

Favour groups L for inclusion in compounds of formula (I) include those of the formula —(CH₂)_{q}— where q is 3, 4, 5 or 6.

Apt groups Ar₁ for inclusion in compounds of the formula (I) include:

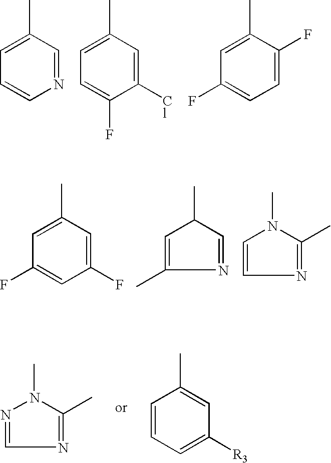

where R³ is hydrogen, methyl, ethyl, fluorine, chlorine, bromine, cyano, methoxyl, methoxylmethyl, trifluoromethoxy, methylthio, ethylthio or methylsulforyl.

A particularly apt group Ar¹ for inclusion in compounds of the (III) is the 3-fluoro-phenyl group.

Favoured compounds of formula (III) include:
7-((R)-2-{4-[8-(3-fluorophenyl)-[1,7]naphthyridin-6-yl]-butylamino}-1-hydroxyethyl)-4-hydroxy-3H-benzothiazol-2-one;
7-((R)-2-{3-[8-(3-fluorophenyl)-[1,7]naphthyridin-6-yl]-propylamino3-1-hydroxy-ethyl)-4-hydroxy, -3H-benzothiazol-2-one;
   and pharmaceutically acceptable salts thereof.

One sub-set of compounds of the formula (I) possessing β₂-adrenergic receptor and M₃ receptor inhibiting properties are these of the formula (IV):

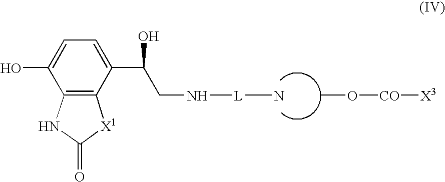

wherein X₁, L,

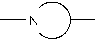

and X₃ are as defined in relation to formula (I).

In compounds of formula (IV) a particularly apt value for X¹ is sulphur.

In compounds of formula (IV) X³ is aptly a group of the formula C(Ar²)(Ar³)(CH₂)ₙOH. Hence apt compounds of formula (IV) include those of the formula (V):

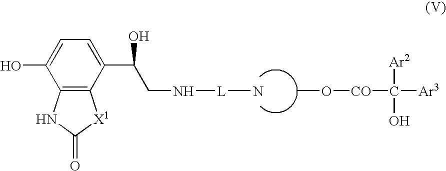

wherein L,

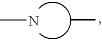

Ar², Ar³ are as defined in relation to formula (I).

Ar² and Ar³ may be independently an optionally substituted phenyl group as defined in relation to formula (I).

In certain apt compounds of formula (V) Ar² is a phenyl group or thienyl group. In certain apt compounds of formula (V) Ar³ is a phenyl group or thienyl group. In certain apt compounds of formula (V), C(Ar²)(Ar³)(CH₂)ₙOH is a group of the formula

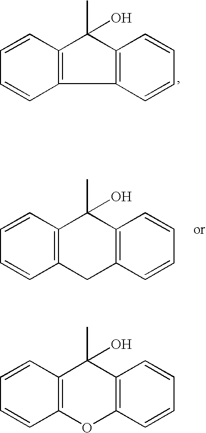

Particularly apt values for

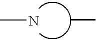

for use in the compounds of formula (V) include

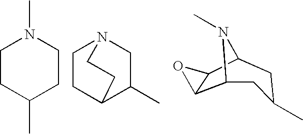

thereby including quaternary compounds where the tertiary amine group shown is alkylated with group NN to give the corresponding quaternary amine. Group NN can be C₁-C₈-alkyl, C₁-C₈-alkaryl, C₁-C₈-alk heteroaryl, CH₂CO-aryl or CH₂CO-heteroaryl.

Particularly apt value for L in the compounds of formula (V) include these set forth in relation to formula (II) and (III).

In alternative compounds of formula the (IV) X³ is aptly a group of the formula NHAr³. Hence alternative apt compounds of the formula IV include those of the formula (VI):

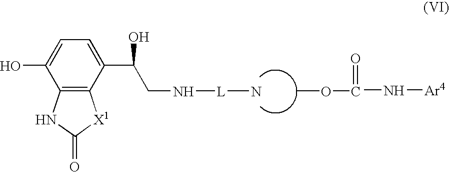

wherein L,

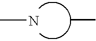

and Ar⁴ are as defined in relation to formula (I) and favourably as defined in relation to formula (IV).

A particularly apt group Ar⁴ is 2-biphenyl.

A particularly suitable group

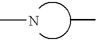

for use in compounds of the formula (VI) is a 4-piperidyl group so that particularly suitable compounds of the formula (VI) include those of the formula (VII)

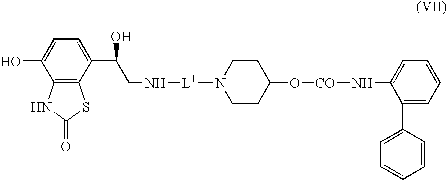

particularly wherein L1 is a group of the formula:

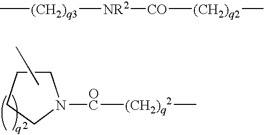

wherein q³ is 0, 1, 2 or 3, q³ is 0, 1, 2 or 3, q⁴ is 1, 2 or 3 and R² is alkyl of 1, 2, 3 or 4 carbon atoms or a hydrogen atom.

Compounds of the formula (VII) include biphenyl-2-yl carbamic acid 1-(2-{(R)-3-[(R)-2-hydroxy-4-hydroxy-2-oxo-2,3-dilydro-benzothiazol-7-yl)-ethylamino]-pyorolidin-1-yl}-2-oxo-ethyl)-piperidia-4-yl ester and pharmaceutically acceptable salts thereof.

Certain specific compounds of the formula (I) are as follows:

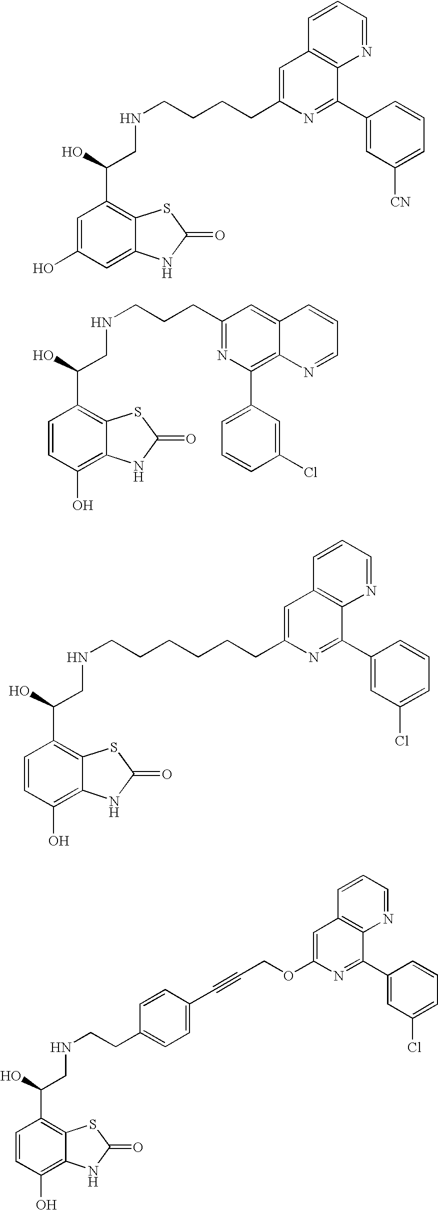

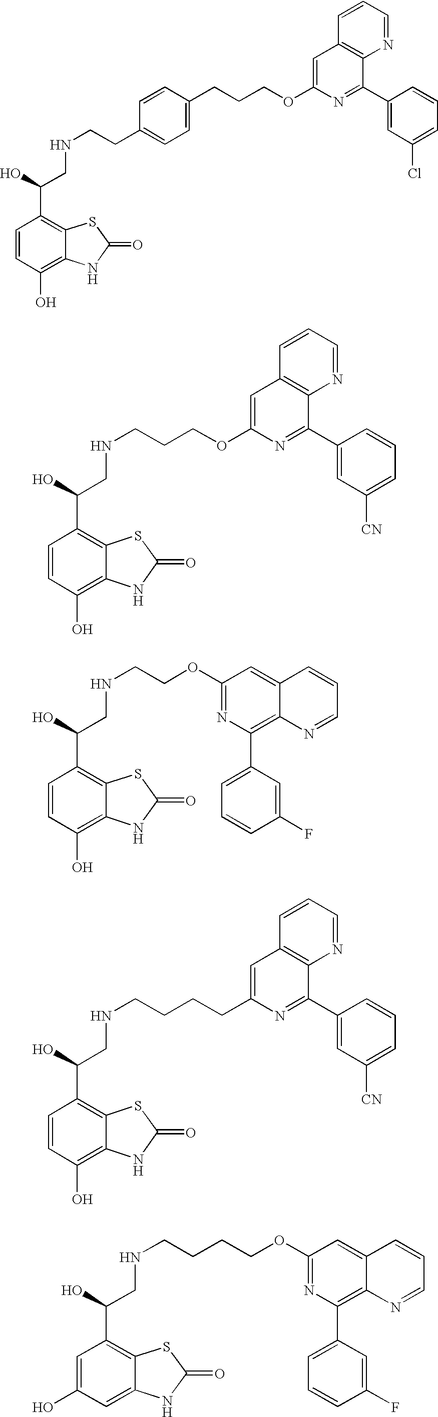

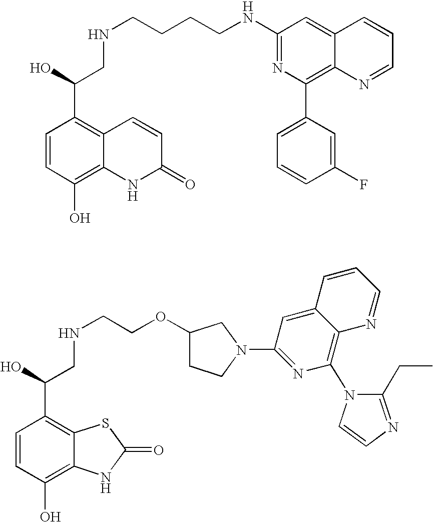

The compounds of the invention may be prepared by known chemical methodology. Thus for example the compounds of formula (I) may be prepared by the reaction of compounds of the formula (VIII) and (IX)

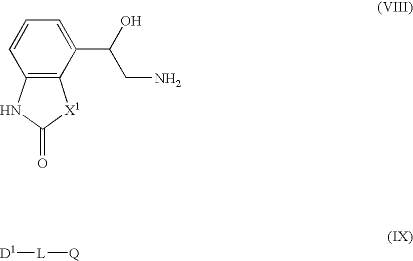

where X¹, L and Q are defined as in relation to formula (I) and D is a displaceable moiety.

When desired any reactive functional groups in the compounds of formulas (VIII) and (IX) may be reversibly protected in conventional manner. This for example the compound of formula (VIII) may be employed as its 4-tert-butoxy-2-isopropoxy analogue and the protecting group removed in conventional manner after the coupling reaction has been performed.

Suitable displaceable groups D¹ include chlorine, bromine, toluenesulfonyl, methylsulfonyl and the like.

A further process for the preparation of the compounds of formula (I) includes the reaction of compounds of the formulas (X) and (XI):

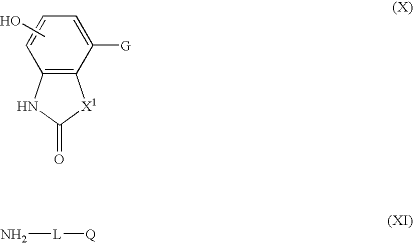

wherein X¹, L and Q are as defined in relation to formula (I) and G is a

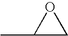

group or a CH(OH)CH₂D group where D is a displaceable moiety.

As outlined above reactive functionalities in the compounds of formulas (X) and (XI) may be reversibly protected as desired. Suitable displaceable moieties are as outlined above.

The preceding coupling reactions may be effected in any convenient solvent such as dimethylsulfoxide, dimethylformamide, acetylnitrile, acetone, tetrahydrofuran or the like. Generally a non-extreme temperature is employed, for example from 10° to 100° C., more conveniently between room temperature and 80° C.

If desired proton acceptor such as a carbonate or bicarbonate may be present, for example Na₂CO₃.

When the above coupling reactions lead to a mixture of optical isomers, these may be separated by conventional techniques, for example fractional crystallisation or column chromatography.

The compounds may be recovered, purified and crystallised in conventional manner.

Isolation of the pure compound from a solvent can lead to the formulation of solvates such as hydrates. Crystalline compounds may be obtained by conventional methods. The free base may be obtained from salts by basification if desired. Salts may be formed by neutralisation of the free base with an acid or by salt exchange in conventional manner.

The compounds of formula (IX) and (XI) may also be prepared by methods known in the art.

Thus for example a compound of formula (IX) or (XI) wherein Q is optionally protected group of formula:

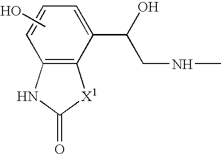

may be prepared by the reaction of a compound of the formula (VIII) is set out above with a compound of the formula (XII)

D¹-L-Q (XII)

where D¹ and D² are displaceable groups. If desired D² may be more readily displaceable than D1.

In cases where the linker group is symmetrical, D² and D³ may be the same.

In a preferred process for the preparation of such symmetric compounds of the formula (IX) or (XI) a diamine of the formula

H₂N-L²-NH₂

where L2 is the residue of the linker group L may be reacted with an optionally protected compound of the formula (X) and thereafter remove the protecting groups if present.

Such reactions can take place in solvents as outlined above and at temperatures as outlined above.

For compounds of formula (I) wherein Q is a

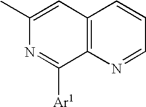

group, the relevant intermediates may be prepared by various processes depending on the nature of the atom directly attached to the naphthyridine residue as follows.

With respect to compounds of the formula (I) wherein there is a carbon atom attached directly to the naphthyridine ring, synthesis may proceed via compounds of the formula

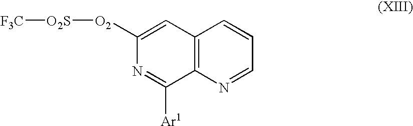

which may be prepared by treatment of the analogous compound of the formula

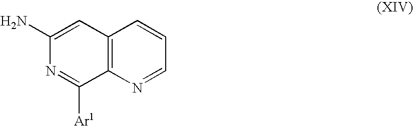

with sodium nitrite in the presence of trifluorosulphonic acid. The reaction may take place initially at a depressed temperature for example 0° C., optionally allowing the reaction to warm to ambient temperature, for example dry tetrahydrofuran.

The compound of formula (XIII) may then be reacted with a compound of the formula (XV)

Br-L³-CN (XVI)

wherein L³ is a residue of a group within the definition of L to yield a compound of the formula

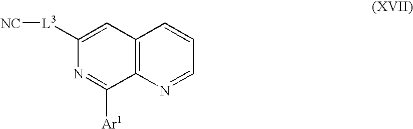

which may be reduced to form a compound of the formula (XVIII):

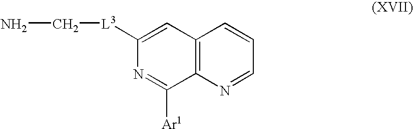

The reaction of the compounds of (XIII) and (XVI) may take place in a dry aprotic solvent such as tetrahydrofuran at a non-extreme temperature.

The resulting compound of the formula (XVII) may be reacted with a compound of the formula (X).

The preparation of a particular compound of formula (I) can be summarised as follows:

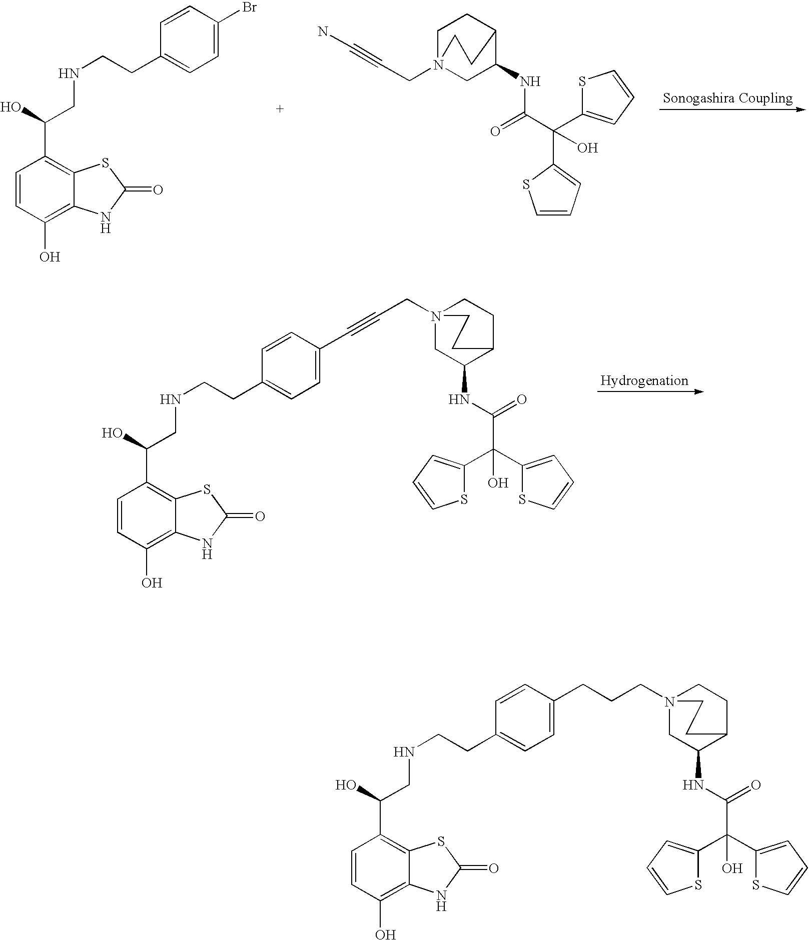

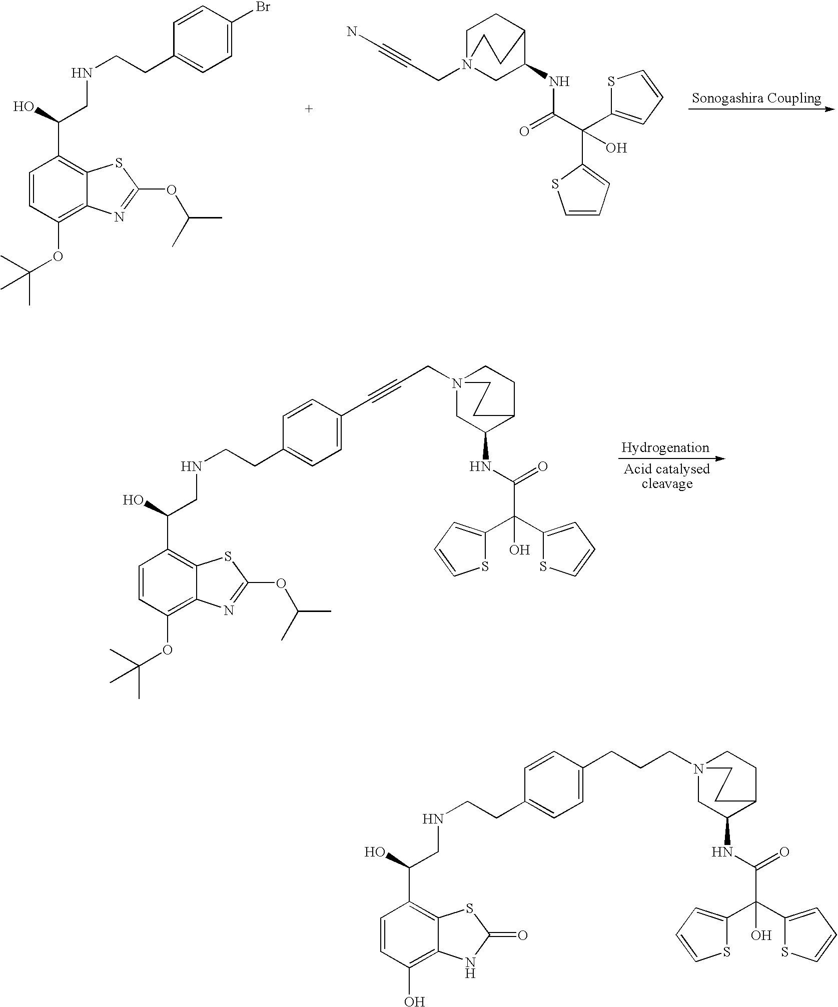

The preparation of compounds having elaborate linking groups may be prepared by preparing suitably functionalised L groups and employing them in processes analogous to those outlined above.

The compounds of the formula (I) are aptly present as pharmaceutically acceptable salts. Suitable salts include those of inorganic and organic acids. Suitable inorganic acids include hydrofluoric hydrochloric, hydrobromic, nitric, sulphuric and phosphoric acids. Suitable organic acids include formic, acetic, trifluoroacetic, propionic, butyric, lactic, citric, tartaric, malic, maleic, succinic, benzoic, p-chloro-benzoic, diphenylacetic, triphenylacetic, o-hydroxybenzoic, p-hydroxybenzoic, 1-hydroxynaphthyl-2-carboxylic, 3-hydroxynaphthyl-2-carboxylic, methanesulphonic, ethanesulfonic, benzenesulphonic and toluenesulphonic acids.

The compounds of formula (I) include at least 1 chiral centre and so exist in individually optically active forms or mixtures thereof e.g. as racemic or diasteriomeric mixtures. R- and S-isomers and mixtures thereof are envisaged by the invention. Aptly the compounds of the formula I are in the form of a pure of substantially pure (>90%, more suitably >95%, preferably greater than 98%). Favourably the compound of the formula (I) has the R configuration at the chiral centre on the hydroxy substituted carbon atom attached to the fused benzene ring shown in formula (I).

Compounds of the invention include at least one chiral centre and therefore the compounds exist in individual optically active isomeric forms or as mixtures thereof, e.g. as racemic or diastereomeric mixtures. Although the invention embraces all isomers and mixtures, it is preferred to use a single isomer, for example the (R) isomer (at the hydroxy substituted carbon atom attached to the benzothiazolone ring shown in formula (I)).

The compounds of formula I, herein “agents of the invention”, are of use in the treatment of adverse medical conditions. Such medical conditions are in general those relating to β₂-adrenergic receptor activation and optional phosphodiesterase (particularly PDE-4) activity and/or M3 muscarinic receptor activity. Hence the compounds of the invention are useful inter alia in the treatment of obstructive or inflammatory airway diseases, for example asthma, bronchoconstriction and the like. Generally in such uses a low incidence of side effects such as tachycardia, tremor and the like is possible. Hence the compounds of the invention may be used on demand (rescue) treatment as well as prophylactic treatment of obstructive or inflammatory airway diseases.

The compounds of formula I in free or salt form, have good β₂-adrenoreceptor agonist activity. The β₂ agonist activity, onset of action and duration of action of the agents of the invention may be tested using the guinea pig tracheal strip in vitro assay according to the procedure of R. A. Coleman and A. T. Nials, J. Pharmacol. Methods (1989), 21(1), 71-86. The binding potency can be measured by a classical filtration binding assay according to the procedure of Current Protocols in Pharmacology (S. J. Enna et al, John Wiley & Son, Inc, 1998), or by cAMP determination in cells expressing β₂-adrenoceptor, according to the procedure of B. January et al, British J. Pharmacol. 123: 701-711 (1998).

The compounds of the invention commonly have a rapid onset of action and have a prolonged stimulating action on the β₂-adrenoceptor, for example having durations of action of the order of up to 24 hours.

Relief of bronchoconstriction can be measured in models such as the in vivo plethysmography models of Chong et al, J. Pharmacol. Toxicol. Methods 1998, 39, 163-168, Hammelmann et al, Am. J. Respir. Crit. Care Med., 1997, 156, 766-775 and analogous models. The compounds of formula I are therefore useful in the treatment of obstructive or inflammatory airways diseases.

In view of their long duration of action, it is possible to administer compounds of formula I once-a-day in the treatment of such diseases. In another aspect, agents of the invention commonly exhibit characteristics indicating a low incidence of side effects commonly encountered with β₂ agonists such as tachycardia, tremor and restlessness, such agents accordingly being suitable for use in on demand (rescue) treatment as well as prophylactic treatment of obstructive or inflammatory airways diseases. The incidence of side effects may be determined, for example, as described by J. R. Fozard et al., Pulmonary Pharmacology & Therapeutics (2000) 14, 289-295.

The affinity (Kᵢ) of compounds of formula I at the human muscarinic acetylcholine M3 receptor can be determined in a competitive filtration binding assay with the radio-labelled antagonist [³H] n-methyl scopolamine methyl chloride (NMS): Membranes prepared from CHO cells stably transfected with human M3 receptor at 10 μg protein/well are incubated with serial dilutions of the agents of the invention, [³H]NMS (0.25 nM) and assay buffer (20 mM HEPES, 1 mM MgCl₂ at pH 7.4) for 17 hours at room temperature. The assay is carried out in a 250 μL final volume, in the presence of a final dimethyl sulfoxide concentration of 1%. Total binding of [³H]NMS is determined in the absence of the agents of the invention with a corresponding substituted volume of assay buffer. Non-specific binding of [³H]NMS is determined in the presence of 300 nM ipratropium bromide. Following the incubation period, the membranes are harvested onto a Unifilter™ GF/B filter plate containing 0.05% polyethyleneimine, using a Brandel™ filtration harvester 9600. Filter plates are dried for two hours at 35° C. before the addition of Microscint™ ‘O’ cocktail, and read on a Packard Topcount™ scintillator using a ³H-Scintillation protocol. All IC50s are calculated with the aid of XL-Fit graph package and Kᵢ values derived using the Cheng-Prusoff correction (Cheng Y., Prusoff W. H. (1973) Biochem. Pharmacol 22 3099-3109).

A PDE-4 inhibitor is a substance or agent that exhibits cyclic nucleotide phosphor-diesterase (PDE) isoenzyme inhibiting activity, selective for type 4 isoenzyme. Such substances possess anti-inflammatory, anti-airways hyperreactivity and bronchodilator properties. They can also possess immunosuppressive and TNFα secretion inhibitory activities. PDE-4 inhibition activity may be measured using the PDE-4 isoenzyme inhibition assay described in WO 03/39544.

Inflammatory or obstructive airways diseases to which the present invention is applicable include asthma of whatever type or genesis including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant from any previously administered symptomatic asthma therapy.

Other inflammatory or obstructive airways diseases and conditions to which the present invention is applicable include adult/acute respiratory distress syndrome (ARDS), chronic obstructive pulmonary or airways disease (COPD or COAD), including chronic bronchitis, or dyspnea associated therewith, emphysema, as well as exacerbation of airways hyperactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, cystic fibrosis, ptilosis, siderosis, silicosis, tabacosis and byssinosis.

With respect to compounds of the invention possessing antimuscarinic activity, they are also useful in the treatment of a condition requiring relaxation of smooth muscle of the uterus, bladder or vascular system. Thus they are thus useful for the prevention or alleviation of premature labour pains in pregnancy. They are also useful in the treatment of chronic and acute urticaria, psoriasis, allergic conjunctivitis, actinitis, rhinitis including allergic rhinitis, mastocytosis, urinary disorders such as urinary incontinence (particularly that caused by an overactive bladder) pollakiuria, neurogenic or unstable bladder, cytospasm and chronic cystitis; gastrointestinal disorders such as irritable bowel syndrome, spastic colitis, diverticulitis and peptic ulceration; and cardiovascular disorders such as vagally induced sinus bradycardia, as well as in ophthalmic interventions

In accordance with the foregoing, the present invention also provides a method for the treatment of an obstructive or inflammatory airways disease which comprises administering to a subject, particularly a human subject, in need thereof a compound of formula (I), or a pharmaceutically acceptable salt thereof as hereinbefore described.

In another aspect, the invention provides a compound of formula (I), or a pharmaceutically acceptable salt thereof as hereinbefore described for use in the preparation of a medicament for the treatment of an obstructive or inflammatory airways disease.

The agents of the invention are also useful as co-therapeutic agents for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Such anti-inflammatory drugs include steroids, in particular glucocorticosteroids such as budesonide, beclamethasone, fluticasone, ciclesonide or mometasone, or steroids described in WO 02/88167, WO 02/12266, WO 02/100879 or WO 02/00679 (especially those of Examples 3, 11, 14, 17, 19, 26, 34, 37, 39, 51, 60, 67, 72, 73, 90, 99 and 101), and non-steroidal steroid agonists such as those described in WO 00/00531, WO 02/10143, WO 03/082280, WO 03/082787, WO 03/104195 and WO 04/005229; LTB4 antagonists such as BIIL 284, CP-195543, DPC11870, LTB4 ethanolamide, LY 293111, LY 255283, CGS025019C, CP-195543, ONO-4057, SB 209247 and SC-53228, and those described in U.S. Pat. No. 5,451,700 and WO 04/108720; LTD4 antagonists such as montelukast, pranlukast, zafirlukast, accolate, SR2640, Wy-48,252, ICI 198615, MK-571, LY-171883, Ro 24-5913 and L-648051; dopamine receptor agonists such as cabergoline, bromocriptine, ropinirole and 4-hydroxy-7-[2-[[2-[[3-(2-phenylethoxy)-propyl]-sulfonylethyl]amino]ethyl]-2(3H)-benzothiazolone and pharmaceutically acceptable salts thereof (the hydrochloride being Viozan®-AstraZeneca); PDE-4 inhibitors such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo) and GRC 3886 (Oglemilast, Glenmark), and those described in WO 92/19594, WO 93/19749, WO 93/19750, WO 93/19751, WO 98/18796, WO 99/16766, WO 01/13953, WO 03/39544, WO 03/104204, WO 03/104205, WO 04/000814, WO 04/000839 and WO 04/005258 (Merck), WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607, WO 04/037805, WO 04/063197, WO 04/103998, WO 04/111044, WO 05/012252, WO 05012253, WO 05/013995, WO 05/030212, WO 05/030725, WO 05/087744, WO 05/087745, WO 05/087749 and WO 05/090345; A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; and A2b antagonists such as those described in WO 02/42298 and WO 03/042214.

Such bronchodilatory drugs include beta-2 adrenoceptor agonists. Suitable beta-2 adrenoceptor agonists include albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 00/75114, which document is incorporated herein by reference, preferably compounds of the Examples thereof, especially a compound of formula

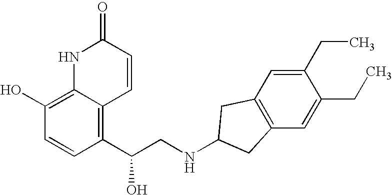

and pharmaceutically acceptable salts thereof, as well as compounds (in free or salt or solvate form) of formula I of WO 04/16601, and also compounds of EP 147719, EP 1440966, EP 1460064, EP 1477167, EP 1574501, JP 05025045, JP 2005187357, US 2002/0055651, US 2004/0242622, US 2004/0229904, US 2005/0133417, US 2005/5159448, US 2005/5159448, US 2005/171147, US 2005/182091, US 2005/182092, US 2005/209227, US 2005/256115, US 2005/277632, US 2005/272769, US 2005/239778, US 2005/215542, US 2005/215590, US 2006/19991, US 2006/58530, WO 93/18007, WO 99/64035, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083, WO 04/80964, WO 04/087142, WO 04/89892, WO 04/108675, WO 04/108676, WO 05/33121, WO 05/40103, WO 05/44787, WO 05/58867, WO 05/65650, WO 05/66140, WO 05/70908, WO 05/74924, WO 05/77361, WO 05/90288, WO 05/92860, WO 05/92887, WO 05/90287, WO 05/95328, WO 05/102350, WO 06/56471, WO 06/74897 or WO 06/8173.

Such bronchodilatory drugs also include anticholinergic or antimuscarinic agents, in particular ipratropium bromide, oxitropium bromide, tiotropium salts, glycopyrrolate, CHF 4226 (Chiesi) and SVT-40776, and also those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/18422, WO 04/05285, WO 04/96800, WO 05/77361 and WO 06/48225.

Suitable antihistaminic/anti-allergic drug substances include acetaminophen, activastine, asternizole, azelastin, bamipin, cetirizine hydrochloride, cexchloro-pheniramine, chlorophenoxamine, clemastine fumarate, desloratidine, dimenhydrinate, dimetinden, diphenhydramine, doxylamine, ebastine, emedastin, epinastine, fexofenadine hydrochloride, ketotifen, levocabastin, loratidine, meclizine, mizolastine, pheniramine, promethazine and tefenadine, as well as those disclosed in JP 2004107299, WO 03/99807 and WO 04/026841 (including any pharmacologically acceptable acid addition salts thereof which may exist).

The agents of the invention may be administered by any appropriate route, e.g. orally, for example in the form of a tablet or capsule; parenterally, for example intravenously; topically to the skin, for example in the treatment of psoriasis; intranasally, e.g. in the treatment of hay fever; or, preferably, by inhalation, particularly in the treatment of obstructive or inflammatory airways diseases. In particular, the agents of the invention may be delivered as an inhalable formulation for the treatment of COPD and asthma.

In a further aspect, the invention also provides a pharmaceutical composition comprising a compound of formula (I) in free form or in the form of a pharmaceutically acceptable salt optionally together with a pharmaceutically acceptable carrier therefor. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture e.g. magnesium stearate, typically 0.05-1.5%. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention also includes (A) a compound of formula I as hereinbefore described in free form, or a pharmaceutically acceptable salt thereof, in inhalable form; (B) an inhalable medicament comprising such a compound in inhalable form together with a pharmaceutically acceptable carrier in inhalable form; (C) a pharmaceutical product comprising such a compound in inhalable form in association with an inhalation device; and (O) an inhalation device containing such a compound in inhalable form.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The following non-limiting Examples illustrate the inventions:

### EXAMPLES

### Example 1

### 7-((R)-2-{4-[8-(3-Fluoro-phenyl)-[1,7]naphthyridin-6-yl]-butylamino}-1-hydroxy-ethyl)-4-hydroxy-3H-benzothiazol-2-one trifluoroacetic acid

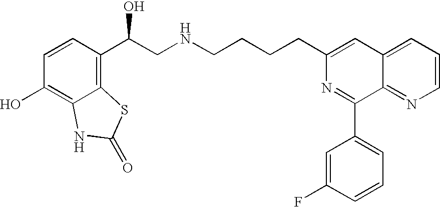

(R)-1-(4-tert-butoxy-2-isopropoxy-benzothiazol-7-yl)-2-{4-[8-(3-fluoro-phenyl)-[1,7]naphthyridin-6-yl]-butylamino}-ethanol trifluoroacetic acid (28 mg, 0.047 mmol) is dissolved in formic acid (3 ml). The reaction mixture is stirred at room temperature for 3 days. The crude product is purified by flash column chromatography (C18, 0% MeCN to 100% MeCN in H₂0+0.1% trifluoroacetic acid as a gradient). To give the title compound. MS (ES+) m/e 505.19 (MH⁺) LCTB09680

### Example 2

### 7-((R)-2-{3-[8-(3-Fluoro-phenyl)-[1,7]naphthyridin-6-yl]-propylamino}-1-hydroxy-ethyl)-4-hydroxy-3H-benzothiazol-2-one trifluoroacetic acid

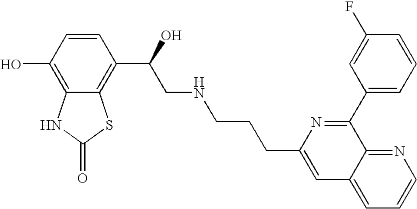

The title compound is prepared from (R)-1-(4-tert-butoxy-2-isopropoxy-benzothiazol-7-yl)-2-{3-[8-(3-fluoro-phenyl)-[1,7]naphthyridin-6-yl]-propylamino}-ethanol using procedure analogous to Example 1. MS (ES+) m/e 491.22 (MH⁺) LCTB09195.

### Example 3

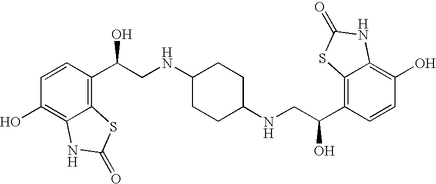

This compound is prepared from (R)-1-(4-tert-butoxy-2-isopropoxy-benzothiazol-7-yl)-2-{4-[(R)-2-(4-tert-butoxy-2-isopropoxy-benzothiazol-7-yl)-2-hydroxy-ethyl-amino]-cyclohexylamino}-ethanol using procedure analogous to Example 1. MS (ES+) m/e 533.37 (MH⁺) PLCb58282.

### Example 4

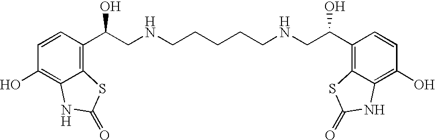

This compound is prepared from (R)-1-(4-tert-butoxy-2-isopropoxy-benzothiazol-7-yl)-2-{5-[(R)-2-(4-tert-butoxy-2-isopropoxy-benzothiazol-7-yl)-2-hydroxy-ethylamino]-pentylamino}-ethanol using procedure analogous to Example 1. MS (ES+) m/e 260.25 (½MH⁺) LCTB07786.

### Example 5

### Biphenyl-2-yl-carbamic acid 1-(2-{(R)-3-[(R)-2-hydroxy-2-(4-hydroxy-2-oxo-2,3-dihydro-benzothiazol-7-yl)-ethylamino]-pyrrolidin-1-yl}-2-oxo-ethyl)-piperidin-4-yl ester trifluoroacetic acid

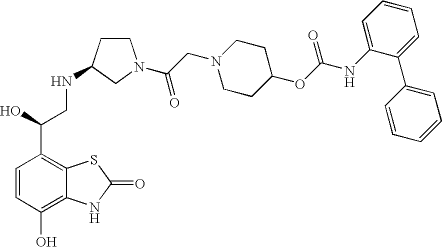

Biphenyl-2-yl-carbamic acid 1-(2-{(R)-3-[(R)-2-(4-tert-butoxy-2-isopropoxy-benzo-thiazol-7-yl)-2-hydroxy-ethylamino]-pyrrolidin-1-yl}-2-oxo-ethyl)-piperidin-4-yl ester trifluoro-acetic acid (27 mg, 0.032 mmol) is dissolved in ⁱPrOH (2 ml) and (2M) HCl_{(aq)} (1 ml). The reaction mixture is heated to 80° C. for 48 hours. The title compound is obtained by flash column chromatography (C18, 0% MeCN to 100% MeCN in H₂0+0.1% trifluoroacetic acid as a gradient). MS (ES+) m/e 632.24 (MH⁺) LCTB13053

### Preparation 1

### (R)-1-(4-tert-Butoxy-2-isopropoxy-benzothiazol-7-yl)-2-{4-[8-(3-fluoro-phenyl)-[1,7]naphthyridin-6-yl]-butylamino}-ethanol trifluoroacetic acid

4-[8-(3-Fluoro-phenyl)-[1,7]naphthyridin-6-yl]-butylamine trifluoroacetic acid (25.8 mg, 0.087 mmol) is dissolved in dimethylformamide (0.5 mL) with bis(trimethyl-silyl)acetamide (0.011 mL, 0.044 mmol). The reaction mixture is stirred at room temperature for 30 minutes. 4-tert-Butoxy-2-isopropoxy-7-(R)-oxiranyl-benzothiazole (WO 2004/016601) (26.8 mg, 0.087 mmol) is dissolved in Dimethylformamide (0.5 mL) and is added to the reaction mixture. The reaction mixture is heated to 80° C. for 18 hours. The title compound is obtained by flash column chromatography (C18, 0% MeCN to 100% MeCN in H₂0+0.1% trifluoroacetic acid as a gradient). MS (ES+) m/e 603.35 (MH⁺) LCTB09559

### Preparation 2

### 8-(3-Fluoro-phenyl)-[1,7]naphthyridin-6-yl]-butylamine trifluoroacetic acid

4-[8-(3-Fluoro-phenyl)-[1,7]naphthyridin-6-yl]-butyronitrile (460 mg, 1.58 mmol) is dissolved in dry tetrahydrofuran (19 mL). LiAlH₄ (3.2 mL, 3.2 mmol) is added proportion wise at 0° C. over a 3 hour period. The reaction mixture is quenched with methanol and is stirred at room temperature for 10 minutes. The reaction mixture was reduce and the title compound is obtained by flash column chromatography (C18, 0% MeCN to 100% MeCN in H₂0+0.1% trifluoroacetic acid as a gradient). MS (ES+) m/e 296.18 (MH⁺) LCTB09158

### Preparation 3

### 4-[8-(3-Fluoro-phenyl)-[1,7]naphthyridin-6-yl]-butyronitrile

Bu₄NI (1.48 g, 4.03 mmol), Pd(dba)₂ (56 mg, 0.098 mmol), (1,1-bis(diphenyl-phosphine)ferrocene) (52 mg, 0.094 mmol) and trifluoro-methanesulfonic acid 8-(3-fluoro-phenyl)-[1,7]naphthyridin-6-yl ester (0.5 g, 1.34 mmol) are dissolved in tetrahydrofuran (3.9 mL) and 1-methyl-2-pyrrolidinone (3.9 mL) under argon. A solution of 0.5M 3-cyanopropylzinc bromide in tetrahydrofuran (5.37 mL, 2.69 mmol) is added to the reaction mixture drop wise. The reaction mixture is heated to 40° C. for 75 minutes. The title compound is obtained by two flash column chromatography (Silica, 2:1 iso-hexane:EtOAc) and (C18, 0% MeCN to 100% MeCN in H₂0+0.1% trifluoroacetic acid as a gradient). MS (ES+) m/e 292.14 (MH⁺) LCTB08997

### Preparation 4

### 4-trifluoro-methanesulfonic acid 8-(3-fluoro-phenyl)-[1,7]naphthyridin-6-yl ester

Triflic acid (5.2 mL, 58.4 mmol) is added drop wise to dry dimethylformamide (12 mL) at 0° C. over 30 minutes. The reaction mixture is warmed to room temperature. 8-(3-fluoro-phenyl)-[1,7]naphthyridin-6-ylamine (WO 2004/055013) (2.0 g, 8.35 mmol) is dissolved in dimethylformamide (3 mL) and is added drop wise to the reaction mixture over 30 minutes at room temperature. The reaction mixture is cooled to 0° C. and sodium nitrite (1.15 g, 16.7 mmol) is added over 30 minutes. The reaction mixture is warmed to room temperature and stirred for 60 minutes. The reaction mixture is diluted with EtOAc, washed with water, (2M) NaOH_{(aq)} and brine. Dried over MgSO₄, filtered and reduced in vacuo. The title compound is obtained by flash column chromatography (Silica, 2:1 iso-hexane:EtOAc) MS (ES+) m/e 373.05 (MH⁺) LCTB08837

### Preparation 5

### (R)-1-(4-tert-Butoxy-2-isopropoxy-benzothiazol-7-yl)-2-{3-[8-(3-fluoro-phenyl)-[1,7]naphthyridin-6-yl]-propylamino}-ethanol

The title compound is prepared from 3-[8-(3-fluoro-phenyl)-[1,7]naphthyridin-6-yl]-propylamine using procedure analogous to Preparation 1. MS (ES+) m/e 589.27 (MH⁺) LCTB09037.

### Preparation 6

### 3-[8-(3-Fluoro-phenyl)-[1,7]naphthyridin-6-yl]-propylamine

The title compound is prepared from 3-[8-(3-Fluoro-phenyl)-[1,7]naphthyridin-6-yl]-propionitrile using procedure analogous to Preparation 2. MS (ES+) m/e 282.19 (MH⁺) LCTB08949.

### Preparation 7

### 3-[8-(3-Fluoro-phenyl)-[1,7]naphthyridin-6-yl]-propionitrile trifluoroacetic acid

The title compound is prepared from 2-cyanoethyllzinc bromide using procedure analogous to Preparation 3. MS (ES+) m/e 278.10 (MH⁺) LCTB08897.

### Preparation 8

### (R)-1-(4-tert-Butoxy-2-isopropoxy-benzothiazol-7-yl)-2-{4-[(R)-2-(4-tert-butoxy-2-isopropoxy-benzothiazol-7-yl)-2-hydroxy-ethylamino]-cyclohexylamino}-ethanol

1,4-diaminocyclohexane (46 mg, 0.41 mmol) and bis(trimethylsilyl)acetamide (0.10 ml, 0.41 mmol) are dissolved in dry dimethylformamide (3 mL) and stirred at room temperature for 30 minutes. 4-tert-butoxy-2-isopropoxy-7-(R)-oxiranyl-benzothiazole (WO 2004/016601) (250 mg, 0.0.81 mmol) is dissolved in dry dimethylformamide (1 mL) and is added to the reaction mixture. The reaction mixture is heated to 80° C. for 42 hours. The reaction mixture is reduced and dissolved in dichloromethane. The title compound is precipitated using methanol and filtered. MS (ES+) m/e 729.60 (MH⁺) PLCb58171.

### Preparation 9

### (R)-1-(4-tert-Butoxy-2-isopropoxy-benzothiazol-7-yl)-2-{5-[(R)-2-(4-tert-butoxy-2-isopropoxy-benzothiazo-7-yl)-2-hydroxy-ethylamino]-pentylamino}-ethanol

The title compound is prepared from 1,5-diaminopentane using procedure analogous to Preparation 8. MS (ES+) m/e 359.27 (½MH⁺) LCTB07668.

### Preparation 10

### Biphenyl-2-yl-carbamic acid 1-(2-[{(R)-3-[(R)-2-(4-tert-butoxy-2-isopropoxy-benzothiazol-7-yl)-2-hydroxy-ethylamino]-pyrrolidin-1-yl}-2-oxo-ethyl]-piperidin-4-ester trifluoroacetic acid

Biphenyl-2-yl-carbamic acid 1-[2-((R)-3-amino-pyrrolidin-1-yl)-2-oxo-ethyl]-piperidin-4-yl ester (68 mg, 0.16 mmol) and bis(trimethylsilyl)acetamide (0.011 ml, 0.09 mmol) are dissolved in dry dimethylformamide (1 mL). The reaction mixture is stirred at room temperature for 30 minutes. 4-tert-Butoxy-2-isopropoxy-7-(R)-oxiranyl-benzothiazole (WO 2004/016601) (50 mg, 0.16 mmol) is added to the reaction mixture. The reaction mixture is heated to 80° C. for 18 hours. The title compound is obtained by flash column chromatography (C18, 0% MeCN to 100% MeCN in H₂0+0.1% trifluoroacetic acid as a gradient). MS (ES+) m/e 730.37 (MH⁺) LCTB12774

### Preparation 11

### Biphenyl-2-yl-carbamic acid 1-[2-((R)-3-amino-pyrrolidin-1-yl)-2-oxo-ethyl]-piperidin-4-yl ester

((R)-1-{2-[4-(Biphenyl-2-ylcarbamoyloxy)-piperidin-1-yl]-acetyl}-pyrrolidin-3-yl)-carbamic acid tert-butyl ester (1.17 g, 2.24 mmol) is dissolved in dichloromethane (10 ml). Trifluoroacetic acid (5 ml) is added and the reaction mixture is stirred at room temperature for 2 hours. The reaction mixture is basified using NaHCO₃(aq). The aqueous layer was extracted using dichloromethane (×2). The organics are washed with water, brine and dried over MgSO₄ and reduced in vacuo. To yield the title compound. MS (ES+) m/e 423.20 (MH⁺) LCTB12601

### Preparation 12

### ((R)-1-{2-[4-(Biphenyl-2-ylcarbamoyloxy)-piperidin-1-yl]-acetyl}-pyrrolidin-3-yl)-carbamic acid tert-butyl ester

(R)-{1-[2-(4-Hydroxy-piperidin-1-yl)-acetyl]-pyrrolidin-3-yl}-carbamic acid tert-butyl ester (520 mg, 1.6 mmol) and 2-biphenylisocyanate (630 mg, 1.75 mmol) are dissolved in N-methyl-2-pyrrolidone (2 ml). The reaction mixture is heated to 70° C. over night. Further 2-biphenylisocyanate (300 mg, 0.9 mmol) is added and heated to 70° C. for 5 hours. The reaction mixture was reduce in vacuo and is cleaned by flash column chromatography (C18, 0% MeCN to 100% MeCN in H₂0+0.1% trifluoroacetic acid as a gradient). The MeCN was removed in vacuo. The aqua's is basified using NaHCO₃(aq) and extracted into dichloromethane. The organics are reduced in vacuo to give the title compound. MS (ES+) m/e 523.23 (MH⁺) LCTB12519

### Preparation 13

### ((R)-{1-[2-(4-Hydroxy-piperidin-3-yl)-acetyl]-pyrrolidin-3-yl}-carbamic acid tert-butyl ester

(R)-(3-(boc)amino)-pyrrolidine (1.5 g, 8.1 mmol) and triethylamine (2.3 ml, 16.1 mmol) are dissolved in (dry) tetrahydrofuran (150 ml). Chloroacetyl chloride (0.67 ml, 8.5 mmol) is added dropwise and the reaction mixture is stirred at room temperature for 2 hours. Triethylamine (2.3 ml, 16.1 mmol) and piperidin-4-ol (4.07 g, 40.3 mmol) are added to the reaction mixture. The reaction mixture is heated to 50° C. over night. The title compound is obtained by flash column chromatography (C18, 0% MeCN to 100% MeCN in H₂0+0.1% trifluoroacetic acid as a gradient). MS (ES+) m/e 328.19 (MH⁺) LCTB12207

1. A compound of the formula (I): 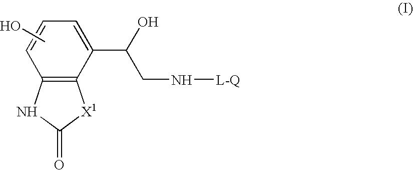
where Q is 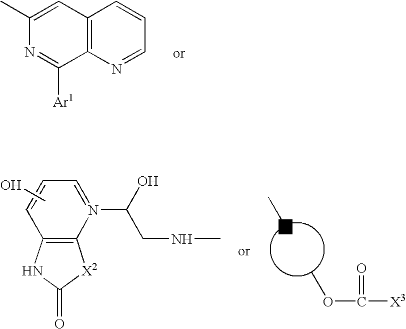
where X³ is 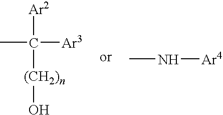
wherein
X¹ and X² are independently selected from S, CH₂CH₂, CH:CH₂ or CH₂0;
Ar¹ is a phenyl, pyridyl, diazinyl or triazinyl group optionally substituted by one or two moieties selected from methyl, ethyl, fluorine, chlorine, bromine, cyano, methoxy, ethoxy, methoxymethyl, trifluoromethyl, trifluoromethoxy, methylthio, ethylthio or methylsulforyl;
Ar² is C₃-C₁₀-cycloalkyl, a thienyl group or a phenyl group optionally substituted by one or two moieties selected from methyl, ethyl, fluorine, chlorine, bromine, cyano, methoxy, ethoxy, methoxymethyl, trifluoromethyl, trifluoromethoxy, methylthio, ethylthio or methylsulforyl; in compounds wherein R¹ is an optionally substituted phenyl group, Ar² may be directly linked to R¹ by a CH₂, CH₂CH₂, CH:CH, OCH₂ or CH₂O group;
Ar³ is C₃-C₁₀-cycloalkyl, a thienyl group or a phenyl group substituted by one or two moieties selected from methyl, ethyl, fluorine, chlorine, bromine, cyano, methoxy, ethoxy, methoxymethyl, trifluoromethyl, trifluoromethoxy, methylthio, ethylthio, methylsulfonyl; in compounds wherein Ar² is an optionally substituted phenyl group R may be linked to Ar² by a CH₂, CH₂CH₂, CH:CH, OCH₂ of CH₂O group.
Ar⁴ is a biphenyl group optionally substituted by one or two moieties selected from fluorine, chlorine, bromine, methyl, ethyl, cyano, methoxy, ethoxy, methoxymethyl, trifluoromethyl, trifluoromethoxy, methylthio, ethylthio or methylsulforyl;
n is 0, 1 or 2;
L is a hydrocarbon linking group of 2 to 20 carbon atoms which may be optionally interrupted by O, N, or CO, and is aptly a group of the form:
Y¹—Y²—Y³
wherein:
Y² is not present or is a group of the formula CH—Y⁴-Q or —N—Y⁴-Q where Q independently is a group as defined above and Y⁴ is a bond or a hydrocarbon linking group of 1 to 8 carbon atoms optionally interrupted by O, NH or CO;
Y¹ is attached to the NH group shown is formula (I) and is a hydrocarbon linking group of 2 to 20 carbon atoms optionally containing an ether oxygen atom or;
Y³ is not present or is selected from 0 or a group NR² where R² is a hydrogen atom or an alkyl group of 2 to 3 carbon atoms optionally linked to a carbon atom within Y¹ or Y² to form a ring or 4, 5, 6 or 7 ring atoms;
and 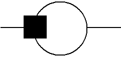
represents a group of the formula: 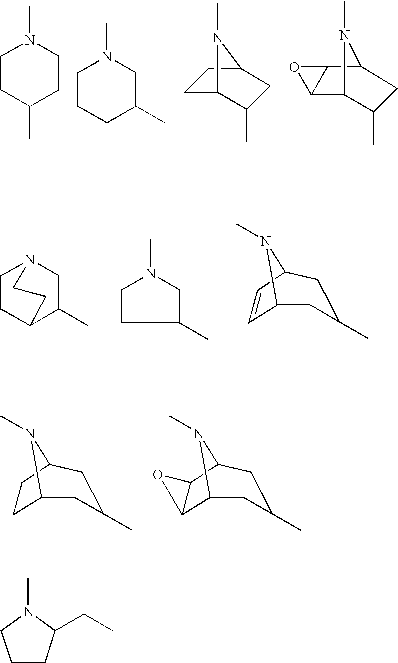
or a pharmaceutically acceptable salt thereof.

2. A compound as claimed in claim 1 of the formula (II) 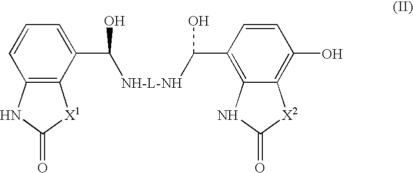
wherein X¹, X² and L are as defined in claimed 1.

3. A compound as claimed in claim 1 of the formula (III): 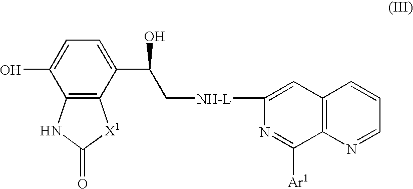
wherein X¹, L and Ar¹ are as defined in claim 1.

4. A compound as claimed in claim 1 of the formula (IV): 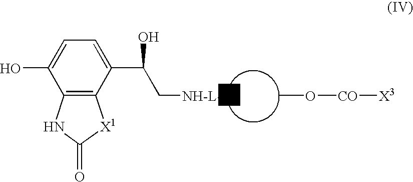
wherein X¹, L, 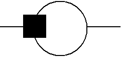
and X³ are as defined in claim 1.

5. A compound as claimed in claim 1 of the formula (V): 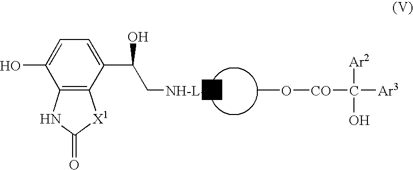
wherein X¹, L, 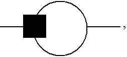
Ar², Ar³ are as defined in claim 1.

6. A compound as claimed in claim 5 wherein Ar² and Ar³ are either both phenyl groups or both thienyl groups.

7. A compound as claimed in claim 1 that is
7-((R)-2-{4-[8-(3-Fluoro-phenyl)-[1,7]naphthyridin-6-yl]-butylamino}-1-hydroxy-ethyl)-4-hydroxy-3H-benzothiazol-2-one trifluoroacetic acid;
7-((R)-2-{3-[8-(3-Fluoro-phenyl)-[1,7]naphthyridin-6-yl]-propylamino}-1-hydroxy-ethyl)-4-hydroxy-3H-benzothiazol-2-one trifluoroacetic acid; or
Biphenyl-2-yl-carbamic acid 1-(2-{(R)-3-[(R)-2-hydroxy-2-(4-hydroxy-2-oxo-2,3-dihydro-benzothiazol-7-yl)-ethylamino]-pyrrolidin-1-yl}-2-oxo-ethyl)-piperidin-4-yl ester trifluoroacetic acid.

8. A pharmaceutical composition which comprises a compound as claimed in any one of claims 1 to 7 and a pharmaceutically acceptable carrier thereof.

9. A compound as claimed in any one of claims 1 to 7 for use of a medicament.

10. The use of a compound as claimed in any one of claims 1 to 7 in the manufacture of a medicament for the treatment of a disease amenable to treatment with a β₂ adrenoreceptor agonist and optionally amenable to treatment with PDE-4 inhibitor and/or a M3 muscarinic antagonist.

11. The use as claimed in claim 10 wherein the disease is asthma or chronic obstructive pulmonary disease.

12. A process for the preparation of a compound as claimed in claim 1 which comprises either
(a) the reaction of compounds of formula (VIII) and (IX): 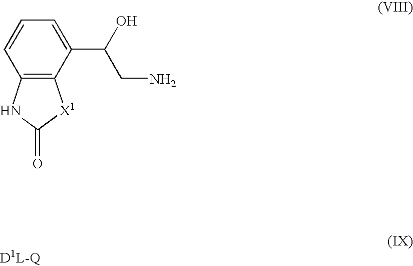
where X¹, L and Q are as defined in claim 1 and D is a displaceable moiety; or
(b) the reaction of compounds of formula (X) and (XI): 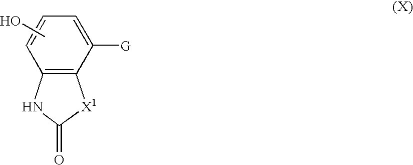
wherein X¹, L and Q are as defined in claim 1 and G is a 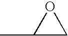
group or a CH(OH)CH₂D group where D is a displaceable moiety.
